(19) United States
(12) Patent Application Publication
(10) Pub. No.: US 2022/0280701 A1
(43) Pub. Date: Sep. 8, 2022

(54) Self-Enclosed Negative Pressure Wound Therapy Device

(71) Applicant: April Corella, Moreno Valley, CA (US)

(72) Inventor: April Corella, Moreno Valley, CA (US)

(21) Appl. No.: 17/190,946

(22) Filed: Mar. 3, 2021

(51) Int. Cl. A61M 1/00; A61F 13/02

(52) U.S. Cl. CPC A61M 1/009; A61F 13/0216; A61M 2205/3331; A61F 2013/00319

(57) ABSTRACT

The described invention is a negative pressure wound therapy device designed for easy application to small to large wounds. The negative pressure device piece applies a vacuum to a wound and absorbs excess exudate or moisture. The negative pressure device comprises a sensor, battery, and small PCBA to monitor the pressure in the dressing and to alert a user when the dressing needs to be changed.

### BACKGROUND OF THE INVENTION

### 1. Field of Invention

The present disclosure relates to wound therapy devices and, more specifically, a wound dressing device that creates a controlled vacuum used to speed the treatment and healing of wounds.

### 2. Description of Related Art

The treatment of wounds predates recorded history. Some cultures, such as the Egyptians, would tend to wounds by applying an antibacterial ointment and then applying and arranging bandages. This practice slowly expanded beyond the Egyptian empire and was adopted by the Greeks, Romans, and Persians, all of whom developed the field of medicine.

In the 19th century, Joseph Lister recognized that antisepsis prevented infection while increasing the healing process. Lister practiced placing carbolic acid onto wounds and increased the rate at which he changed dressings surrounding the wounds. He noticed that the infection rate and gangrene of the wounds treated with his technique were substantially lower than those treated with the previous techniques. During World War I and World War II, the field of wound treatment expanded. Certain techniques like hyperbaric oxygen therapy, laser therapy, hydrotherapy, and electrotherapy were developed. Not all techniques could be applied to all wounds or injuries equally. Acute or persistent wounds needed a new modality for treatment.

In the 1990s, the first Negative Pressure Wound Therapy (“NPWT”), the Food and Drug Administration approved the first device. NPWT devices apply a zone sub-atmospheric pressure within the device and the wound area. NPWT devices treat a wound by placing a dressing over a wound that creates a moist wound environment while managing exudates and removing extracellular fluid from the wound when the treated wound is under vacuum conditions. NPWT devices have successfully treated burns, infections, wounds caused by poor circulation, exposed bones, and artificial implants. An NPWT dressing is typically made up of two separate pieces: the bandage or wound dressing and a vacuum source or pump. Currently-available NPWT devices require external, bulky equipment such as the vacuum source or pump.

When a currently available NPWT dressing is applied to a wound, the patient is forced to be near the pump and is limited in their movement. The pumps also contain tubing, which can kink, crack, or break, preventing the vacuum pressure from being applied. Such limitations are cumbersome and prevent the patient from freely participating in basic activities, such as eating and using the restroom.

Prior approaches are deficient for several reasons. For example, previous approaches rely on tubing or devices connecting the vacuum source to the dressing. Such tubing must be repeatedly adjusted, which can create a leak. Adjusting such tubing can also create restrictions and kinks that hinder the airflow within the system and prevent vacuum pressure from being applied to the dressing's interior. Tubing can also be cumbersome to the patient creating discomfort. Other devices are also complicated due to the many pieces or steps needed to apply the dressing to create a waterproof, air-tight seal. Such components and workings thereof are often not intuitive and therefore present hurdles for the every-day consumer or health care professional.

For example, United States Patent Application Publication No. 2011/0004173 to Hu, et al. is deficient at least because it relies on a spring-type suction device to create and regulate the negative pressure zone within the dressing. Hu's suction device is inherently defective because the spring must be constantly reset and, depending on the tension placed on the spring, the amount of vacuum applied will change. Due mainly to such deficiencies, the amount of suction drastically varies, rendering the treatment wildly inconsistent. Additionally, Hu also relies on bulky tubing to supply the vacuum from the pump to the dressing.

In another example, U.S. Pat. No. 9,956,325 to Malhi is similarly deficient because it discloses a pump that is not attached to the drape and relies on bulky and cumbersome tubing to supply vacuum. Additionally, Malhi utilizes a canister to collect and manage exudate and relies on intermediate connections between the vacuum source and the wound dressing, which are prone to leaking and make the apparatus more cumbersome.

In another example, United States Patent Application Publication No. 2009/0125004 to Shen, et al. is similarly deficient because it relies on a canister to manage exudate, tubing to deliver the vacuum from the pump to the dressing, and an external pump to create the vacuum.

In another example, U.S. Pat. No. 9,950,100 to Blott, et al. is similarly deficient because it relies on a canister to manage exudate, tubing to deliver the vacuum from the pump to the dressing, and an external pump to create the vacuum.

In another example, U.S. Pat. No. 8,257,328 to Augustine, et al. is similarly deficient because it relies on a canister to manage exudate, tubing to deliver the vacuum from the pump to the dressing, and an external pump to create the vacuum.

In another example, U.S. Pat. No. 8,663,198 to Buan, et al. is similarly deficient because it relies on a canister to manage exudate, tubing to deliver the vacuum from the pump to the dressing, and an external pump to create the vacuum.

Thus, there is a need for a negative pressure wound therapy device that is self-contained and does not rely on bulky components while providing a customizable and consistent vacuum at the wound site. This need has heretofore remained unsatisfied.

### SUMMARY OF THE INVENTION

The present disclosure overcomes these and other deficiencies of the prior art by providing a wound dressing with a pump enclosed as a single device.

According to exemplary embodiments of the present disclosure, the device comprises a wound dressing with a suction pump located therein. In such an embodiment, a Negative Pressure Wound Therapy Device (“NPWT”) is made from a rigid, semi-rigid, or semi-pliable material, which is then applied directly to the wound or wound area by itself or with additional wound dressings. The NPWT device contains a battery, a sensor, a pump, and a printed circuit board assembly (“PCBA”). The PCBA is programmed to regulate negative pressure in the enclosed wound dressing. The PCBA may be made from a variety of materials (rigid or soft). In some embodiments, the bandage is made out of a polyurethane drape or similar material, which is attached to a multilayer, semi-permeable absorbent pad. The number of layers may vary (e.g., a single layer or a plurality of layers) and may be dictated by the indication or use. In one embodiment, the upper portion of the pad comprises silicone and open-cell foam that is in contact with a final gel layer made out of, for example, polyurethane foam. In other embodiments, a final layer is added that can be comprised of hydrophobic foam or silicone material that covers the wound and periwound site. In such an embodiment, the bandage is temporarily adhered to the patient's skin around the wound and periwound site, sealing the area and allowing the NWPT to create a negative pressure within the bandage. The invention described herein is not limited to human patients. As used herein, the term “patient” refers to any type of living organism and is not limited to a particular species of a living creature.

In an embodiment of the present invention, a negative pressure wound therapy device comprises a dressing, a drape, a contact layer, and a pump attached to the dressing; wherein the pump comprises an air pump, a power source, an air inlet, an air outlet, and a pressure sensor; and wherein the pump creates a region of sub-atmospheric pressure in a region between the drape and a patient.

In another embodiment, the dressing further comprises an intermediate layer disposed between the drape and the contact layer. In another embodiment, the intermediate layer comprises hydrophobic materials. In another embodiment, the intermediate layer comprises hydrophilic materials. In another embodiment, the dressing comprises a pull tab. In another embodiment, the negative pressure wound therapy device further comprises a second pump attached to the dressing, the second pump comprising a second air pump, a second power source, a second air inlet, a second out outlet, and a second pressure sensor. In another embodiment, the pump is attached to the dressing via a connector.

In another exemplary embodiment of the present invention, a method for treating a patent's wound using a negative pressure wound therapy device comprises adhering a dressing to the patient circumscribing the wound area, wherein the dressing comprises a drape covering a contact layer; and drawing air from within the dressing using a pump comprising a pump and power source, wherein the pump is attached to the dressing; wherein the pump draws air through the contact layer. In another embodiment, the dressing further comprises an intermediate layer disposed between the drape and the contact layer, and wherein the pump additionally draws air through an intermediate layer, whereby the intermediate layer captures exudate from the wound. In another embodiment, the drape comprises a pull tab. In another embodiment, the intermediate layer comprises hydrophilic materials. Although the foregoing description is discussed as having a specified number of layers, any number of layers can be utilized without departing from the contemplated embodiments.

In another exemplary embodiment of the present invention, a negative pressure wound therapy device comprises a dressing comprising a drape and a contact layer, and a pump attached to the dressing; wherein the pump comprises an air pump, a power source, an air inlet, and an air outlet, and wherein the pump creates a region of sub-atmospheric pressure in a region proximate the air inlet. In another embodiment, the dressing further comprises an intermediate layer disposed between the drape and the contact layer. In another exemplary embodiment, the intermediate layer comprises hydrophobic materials. In another exemplary embodiment, the intermediate layer comprises hydrophilic materials. In another exemplary embodiment, the device further comprises a pressure sensor. In another exemplary embodiment, the device further comprises a pull tab comprising a first end and a second end, wherein the pull tab is attached to the drape at the first end. In another exemplary embodiment, the device further comprises a second pump attached to the dressing, the second pump comprising a second air pump, a second power source, a second air inlet, a second air outlet, and a second pressure sensor. In another exemplary embodiment, the pump is removably attached to the dressing via a coupling device attached to the drape. In another exemplary embodiment, the drape comprises an adhesive circumscribing its perimeter on a side opposite the pump. In another exemplary embodiment, the device comprises a second pull tab attached to an adhesive release liner, wherein the adhesive release liner is removably attached to the adhesive.

In another exemplary embodiment of the present invention, a method for treating a wound using a negative pressure wound therapy device comprises adhering a dressing to an area circumscribing the wound, wherein the dressing comprises a drape covering a contact layer; and drawing air from within the dressing using a pump comprising an air pump and power source, wherein the pump is attached to the dressing; wherein the pump creates a zone of subatmospheric pressure between the drape and the wound. In another exemplary embodiment, the dressing further comprises an intermediate layer disposed between the drape and the contact layer; and wherein the pump draws air through the intermediate layer, whereby the intermediate layer captures exudate from the wound. In another exemplary embodiment, the drape comprises a pull tab configured to remove the device. In another exemplary embodiment, the intermediate layer comprises hydrophilic materials. In another exemplary embodiment, the pump further comprises a pressure sensor. In another exemplary embodiment, the pump is removably attached to the dressing.

The foregoing and other features and advantages of the invention will be apparent from the following, more particular description of the preferred embodiments of the invention, the accompanying drawings, and the claims.

### BRIEF DESCRIPTION OF THE DRAWINGS

For a more complete understanding of the present disclosure, the objects, and advantages thereof, reference is now made to the ensuing descriptions taken in connection with the accompanying drawings briefly described as follows:

FIG. 1A illustrates an isometric top view of an NPWT device, according to an exemplary embodiment of the present disclosure;

FIG. 1B illustrates a top view of an NPWT device, according to an exemplary embodiment of the present disclosure

FIG. 1C illustrates a bottom view of an NPWT device, according to an exemplary embodiment of the present disclosure;

FIG. 2 illustrates a side exploded detail view of an NPWT device, according to an exemplary embodiment of the present disclosure;

FIG. 3A illustrates an isometric top view of a pump for an NPWT device, according to an exemplary embodiment of the present disclosure;

FIG. 3B illustrates a top view of a pump for an NPWT device, according to an exemplary embodiment of the present disclosure;

FIG. 3C illustrates a bottom view of a pump for an NPWT device, according to an exemplary embodiment of the present disclosure;

FIG. 4 illustrates an isometric detail top view showing interior components of a pump of an NPWT device, according to an exemplary embodiment of the present disclosure;

FIG. 5 illustrates a side detail view showing interior components of a pump of an NPWT device, according to an exemplary embodiment of the present disclosure;

FIGS. 6A-6B illustrate detail views of an NPWT device with a circular drape, according to an exemplary embodiment of the present disclosure;

FIGS. 7A-7B illustrate detail views of an NPWT device with a rectangular drape, according to an exemplary embodiment of the present disclosure; and

FIG. 8 illustrates a top view of an NPWT device with a circular drape and a remotely located pump, according to an exemplary embodiment of the present disclosure.

### DETAILED DESCRIPTION OF EMBODIMENTS

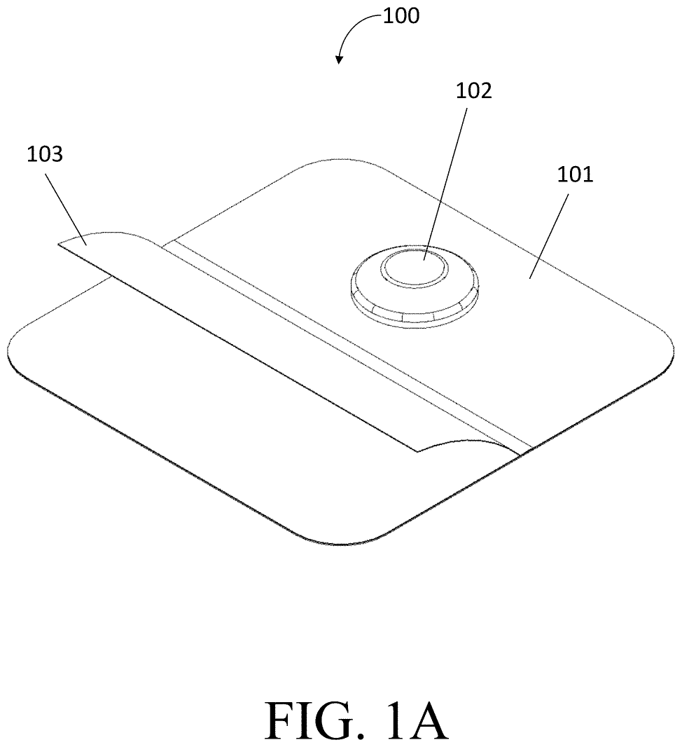

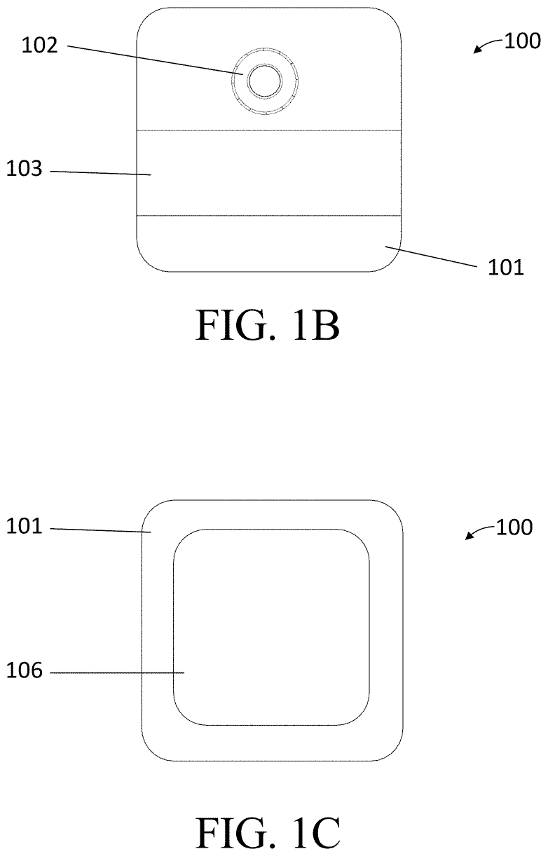

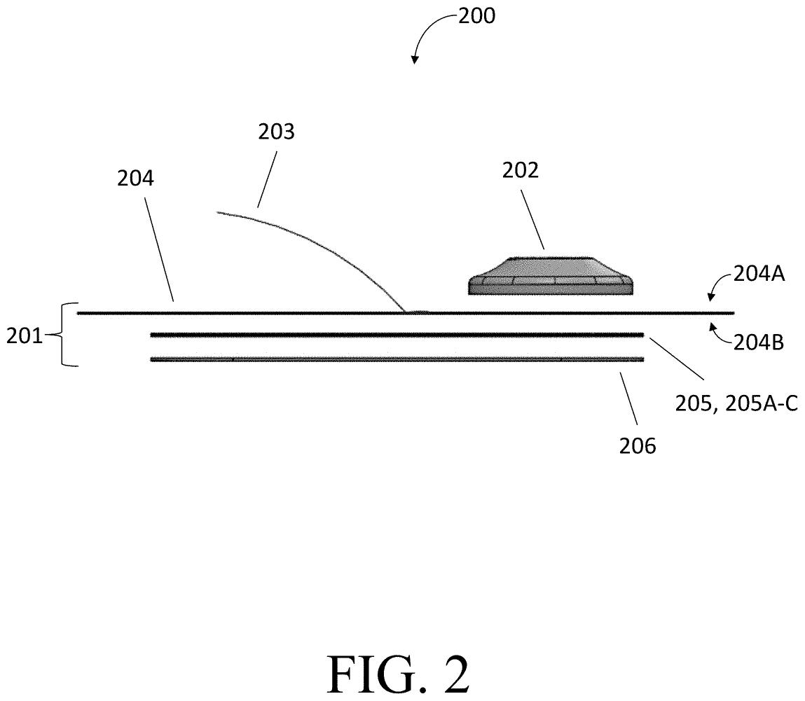

Further features and advantages of the disclosure, as well as the structure and operation of various embodiments of the disclosure, are described in detail below with reference to the accompanying FIGS. 1-8.

In an exemplary embodiment of the present disclosure, the negative pressure wound therapy (“NPWT”) device comprises a dressing and a pump.

In another exemplary embodiment of the present disclosure, the dressing comprises a drape and a wound contact layer. In some embodiments, the dressing further comprises an intermediate layer disposed between the drape and the contact layer. While the dressing may be discussed herein as having a discrete and finite number of layers, any number of layers may be implemented without departing from the contemplated embodiments.

In another exemplary embodiment, the drape comprises a top surface and a bottom surface. In some embodiments, the bottom surface of the drape comprises a skin-safe adhesive. In such an embodiment, the drape is larger than the contact layer and the intermediate layer such that the bottom surface of the drape comprising skin-safe adhesive contacts the patient and holds the NPWT device in place. In other embodiments, the drape and the skin-safe adhesive are configured to create an air-tight seal between the drape and the patient.

In another exemplary embodiment, the wound dressing may comprise several layers that are used to absorb wound exudate. For example, such intermediate layers may comprise a foam layer(s), a gel layer(s), a gauze layer(s), or any number/combination thereof.

In another exemplary embodiment, the contact layer comprises a single layer that is used to absorb exudate and work in conjunction with the vacuum created by the NPWT device. Some embodiments comprise a single contact layer that may comprise a semi-permeable layer, gel layer, hydrophilic, and/or hydrophobic materials (e.g., foam). In some embodiments, the foam contact layer, or foam used in the wound bed, comprises polyurethane or similar material that is used as a contact layer or is used in the wound bed to absorb and/or collect exudate.

In another exemplary embodiment, the contact layer(s) and or/material used in the wound bed comprises hydrocolloid and/or silicone. In some embodiments, a hydrocolloid dressing is an opaque or transparent dressing for wounds and is often biodegradable, breathable, and adheres to a patient's skin so that separate taping is not needed. In another exemplary embodiment, the contact layer(s) and/or material used in the wound bed contains antimicrobial properties, including but not limited to silver, polyhexamethylene biguanide (PHMB), carboxyl methylcellulose, ethylenediamine-tetraacetic acid (EDTA), silver chloride, or any combination thereof. In another exemplary embodiment, the contact layer(s) and/or material used in the wound bed comprises specific materials to manage exudate, for example, sodium alginate. In other embodiments, the contact layer and/or the wound bed materials may comprise biological materials and/or animal-derived materials such as collagen.

In another exemplary embodiment, the NPWT device comprises a pull tab. In some embodiments, the pull tab is attached to the dressing and configured to allow a user to remove the NPWT device from the patient by pulling on the pull tab. In other embodiments, the pull tab is configured such that when a user pulls the pull tab, the adhesive layer is disrupted, allowing the NPWT device to be removed from the patient. In other embodiments, the pull tab is configured to remove a release liner, which, in turn, exposes an adhesive layer. In other words, the pull tab may be configured to easily and quickly remove a storage backing for the adhesive areas. In other embodiments, the NPWT comes sealed in self-contained storage packaging that allows the NPWT to be kept clean while in storage. In such an embodiment, the pull tab may be integrated into the packaging such that when a user removes the packaging, the user also pulls away the backing, exposing the adhesive layer.

In another exemplary embodiment of the present disclosure, the pump comprises a pump, a plurality of sensors, a PCBA, a power source, an inlet port, and an outlet port. In another exemplary embodiment, the pump is operatively connected to an inlet port and an outlet port of the NPWT device. In such an embodiment, the pump is configured to draw air through the inlet port and expel it through the outlet port. In some embodiments, the pump is operatively connected to the PCBA, a power source, and/or the plurality of sensors. In some embodiments, the pump may be configured to create and maintain a vacuum of 0 mmHg to 300 mmHg. Although a specific range of vacuums and/or pressures is shown and described herein, any pressure can be used without departing from the contemplated embodiments. Furthermore, any suitable type of pump may be employed without departing from the embodiments contemplated herein. For example, a dynamic pump may be used. Such pumps include, for example, centrifugal pumps. In another example, positive displacement pumps may be utilized. Such positive displacement pumps include diaphragm pumps, gear pumps, peristaltic pumps, lobe pumps, and piston pumps. As used herein, the terms “vacuum” and “subatmospheric pressure” have similar connotations and scope, both relating to a region where the pressure is lower than ambient pressure or atmospheric pressure in that given region.

In another exemplary embodiment, the plurality of sensors includes a pressure sensor capable of measuring a negative pressure (a vacuum) and a positive pressure. In other embodiments, the plurality of sensors includes but is not limited to temperature sensors, force sensors, accelerometers, potentiometers, humidity sensors, air bubble sensors, and/or position sensors.

In another exemplary embodiment, the PCBA comprises input-output devices, including components for receiving or transmitting information via wired or wireless transmission.

In another exemplary embodiment, the power source is configured to provide power to the components of the NPWT device. In some embodiments, the power source comprises a battery. In such an embodiment, the power source may utilize any type of commercially available battery to power the NPWT device. For example, double-A, triple-A, 9-volt batteries, or any combination thereof, may be used to power the NPWT device. In other embodiments, the NPWT device may utilize a uniquely configured battery to power the NPWT device. In some embodiments, the power source may further utilize a rechargeable battery and/or a photovoltaic cell. In other embodiments, the power source may comprise alternating current (AC) power by, for example, plugging into a 110V wall outlet. The power source may comprise direct current (DC) power by, for example, plugging into a power outlet located within a vehicle.

In another exemplary embodiment of the present disclosure and with reference to FIGS. 1-1C, the NPWT device 100 comprises a dressing 101 and a pump 102. Although pump 102 is shown as being attached to the dressing 101 at a specific location of one side of the dressing 101, pump 102 may be attached to the dressing 101 at any location without departing from the embodiments contemplated herein. Although the NPWT device 100 is shown as comprising a single pump 102, any number of pump assemblies 102 may be utilized without departing from the embodiments contemplated herein. In some embodiments, the NPWT device 100 comprises a pull tab 103. Although the pull tab 103 is shown as being attached to the dressing 101 in the center of dressing 101, the pull tab 103 may be attached to the dressing 101 at any location without departing from the contemplated embodiments.

In some embodiments, the dressing 101 comprises a wound contact layer 106. Although the dressing 101 is shown as having a square shape, any size and/or shape of dressing 101 and/or contact layer 106 may be utilized without departing from the contemplated embodiments. In some embodiments, pump 102 and/or dressing 101 may be configured to allow pump 102 to be reused. In such an embodiment, for example, the dressing 101 may become saturated with exudate. The dressing 101 and contact layer 106 may then be removed and replaced with a new dressing 101 and contact layer 106, and the pump 102 may be transferred to the new dressing 101. Such a configuration utilizes reusability to minimize waste and cost. In other embodiments, the NPWT device 100 is configured such that the dressing 101 and the pump 102 are disposable and are intended for a single-use. Such an embodiment minimizes the possibility that one or more components of the NPWT device 100 become contaminated. Additionally, such an embodiment may be preferred where the patient cannot readily sterilize or disinfect the components or wound, such as on a battlefield. In some embodiments, the dressing 101 may comprise an adhesive that affixes the NPWT device 100 to the patient. In such an embodiment, the adhesive seals the dressing 101 to the patient's skin around the periwound site, enabling the NPWT device 100 to create a negative pressure zone.

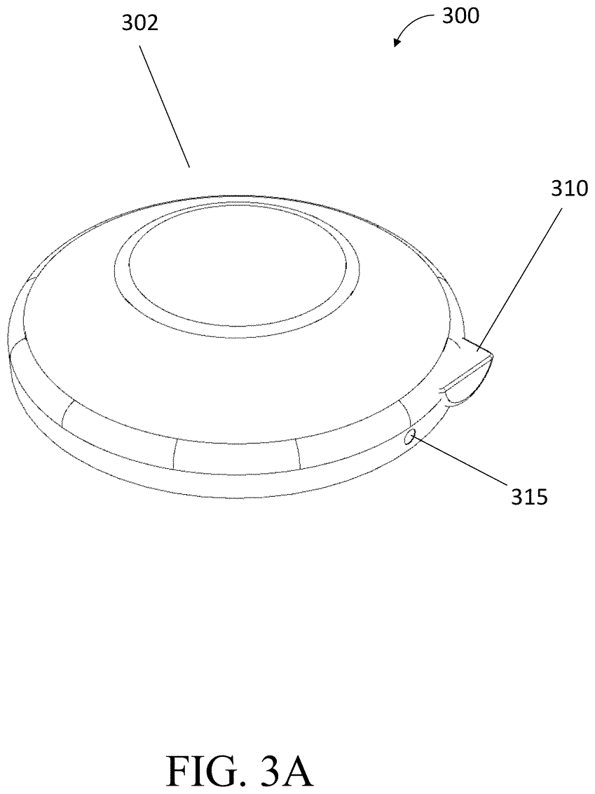

In another exemplary embodiment of the present disclosure and with reference to FIG. 2, the NPWT device 200 comprises a dressing 201 and a pump 202. In some embodiments, dressing 201 comprises a drape 204, an intermediate layer 205, and a contact layer 206. In some embodiments, the drape 204 comprises a top surface 204A and a bottom surface 204B. In some embodiments, the top surface 204A is configured to operably receive the pump 202. In another embodiment, some or all of the bottom surface 204B comprises adhesive. In such an embodiment, the adhesive is configured to hold the NPWT device 200 to the patient and to provide a seal between the drape 204 and the patient's skin (not shown). Exemplary types of adhesive may include but are not limited to cyanoacrylates, UV cured adhesives, epoxy, and medical adhesive silicone. In some embodiments, the adhesive is configured to create an air-tight seal or substantially air-tight seal around the perimeter of the bottom surface 204B, thereby allowing the pump 202 to create and maintain a vacuum. In some embodiments, the wound dressing may be applied by wrapping a film around the dressing 201 in lieu of an adhesive film. The drape 204 may be made from any semi-rigid and/or pliable material that allows the drape 204 to be affixed to the desired location and maintain a waterproof and/or air-tight seal between the NPWT device 200 and the patient's tissue. For example, the drape 204 may comprise polyurethane, vinyl, latex, silicone, or equivalent suitable material may be used, or a combination thereof. In other embodiments, an incomplete seal between the bottom surface 204B and the patient's skin may be preferred. In such an embodiment, the pump will create unidirectional air circulation so that, for example, a care provider can cause air to circulate around a wound site. Such a configuration may also be used to further facilitate exudate flow in a specific, desired manner.

In another embodiment, the intermediate layer 205 comprises a foam layer 205A, a gel layer 205B, and/or a gauze layer 205C. In some embodiments, the foam layer 205A comprises hydrophobic materials configured to pass exudate from the wound area to an absorbent layer, e.g., gauze layer 205C. In other embodiments, the foam layer 205A comprises hydrophilic materials configured to collect and/or absorb exudate. In these embodiments, the foam layer 205A removes exudate and prevents prolonged contact by the exudate with the wound or periwound site. In some embodiments, the foam layer 205A may be excluded entirely, and the NPWT device may comprise a single contact layer 206 or a single type of wound packing material for absorbing and/or collecting wound exudate. In some embodiments, the gel layer 205B is configured to be a barrier against bacteria. In other embodiments, the gel layer 205B is configured to keep moisture in the wound and prevent the wound area from drying out. In some embodiments, the gauze layer 205C is used to protect the wound bed from bacteria, absorb exudate, and/or cover the wound bed.

In another embodiment, the contact layer 206 protects the wound bed and/or periwound area. The contact layer 206 comprises single or multiple layers of permeable, semi-permeable, and/or occlusive dressings comprising silicone, polyurethane, cotton, biological, and/or synthetic polymers, or any combination thereof. The contact layer 206 may be non-adherent and placed on the wound bed to prevent any direct contact between the wound bed and other wound elements such as additional wound dressings or ointments. The contact layer 206 may transmit wound bed fluids such as water vapor to other portions of the dressing 201 or may absorb fluids and wound exudate, for example, by comprising hydrophilic materials. In another exemplary embodiment, the dressing 201 may comprise a pull tab 203. The pull tab 203 may be configured to provide a means for the dressing 201 to be placed over or removed from a wound. In some embodiments, the pull tab 203 may be configured to contact or overlap the top of the dressing 201 or any other suitable location that does not disturb the functions of the NPWT device 200. The pull tab 203 may be made from any rigid, semi-rigid, or pliable material, or a combination thereof. In some embodiments, one or more of the layers 204, 205, 206 may contain medication or other substances/compounds that may, for example, assist in the healing process.

In another exemplary embodiment of the present disclosure, the intermediate layer 205 comprises a plurality of layers 205A-C. One or more of the plurality of layers 205A-C may comprise polyurethane foam, silicone, vinyl, latex, polyurethane, gauze/cotton, collagen, impregnated wound dressing, or a combination thereof. In other embodiments, one or more of the plurality of layers 205A-C comprise a semi-rigid permeable absorbent layer that allows for the absorption of exudates and allows the free-flow of air through the dressing 201. The contact layer 206 provides a sterile environment that may come into contact with exposed portions of a wound. The contact layer 206 may comprise a hydrophobic foam, mesh, gauze, sponge, or any other suitable porous biocompatible material.

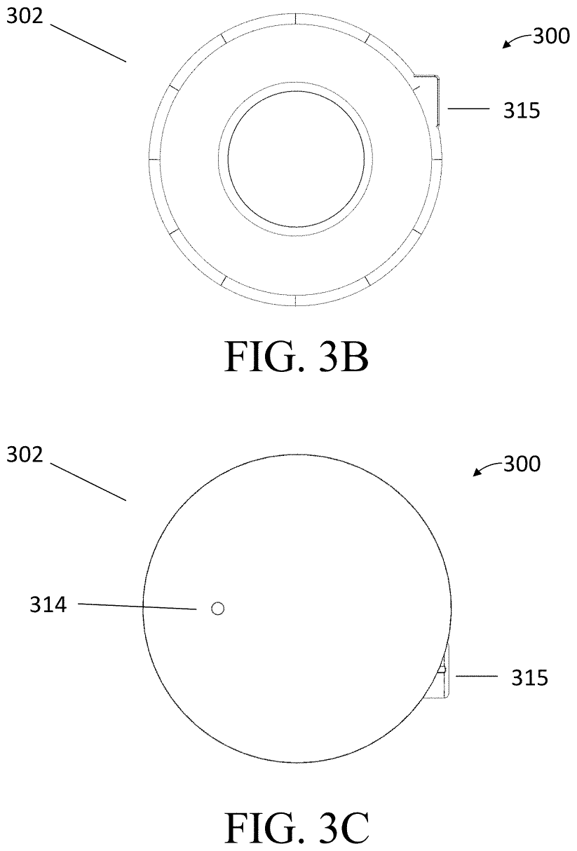

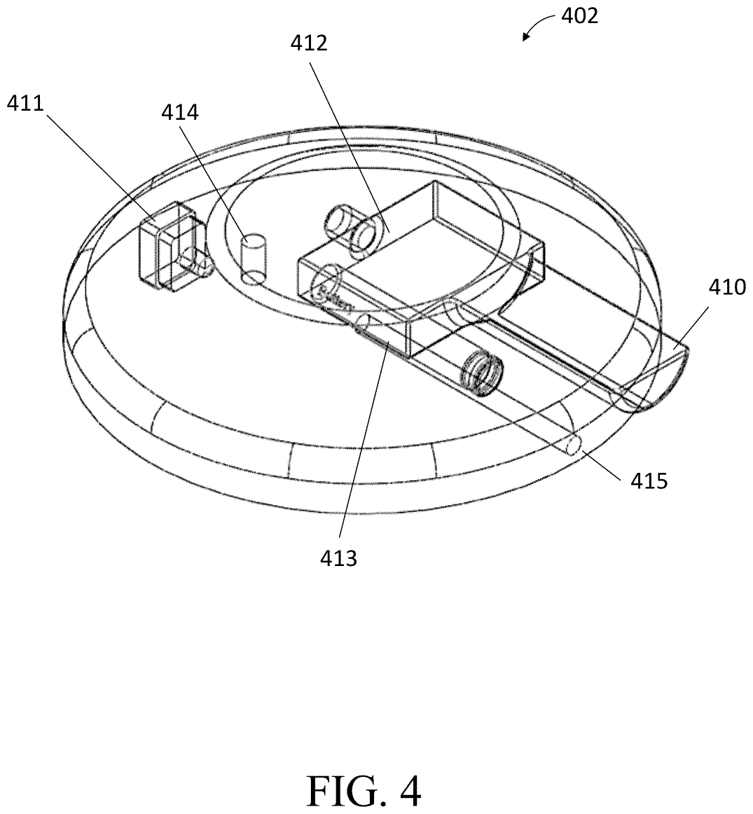

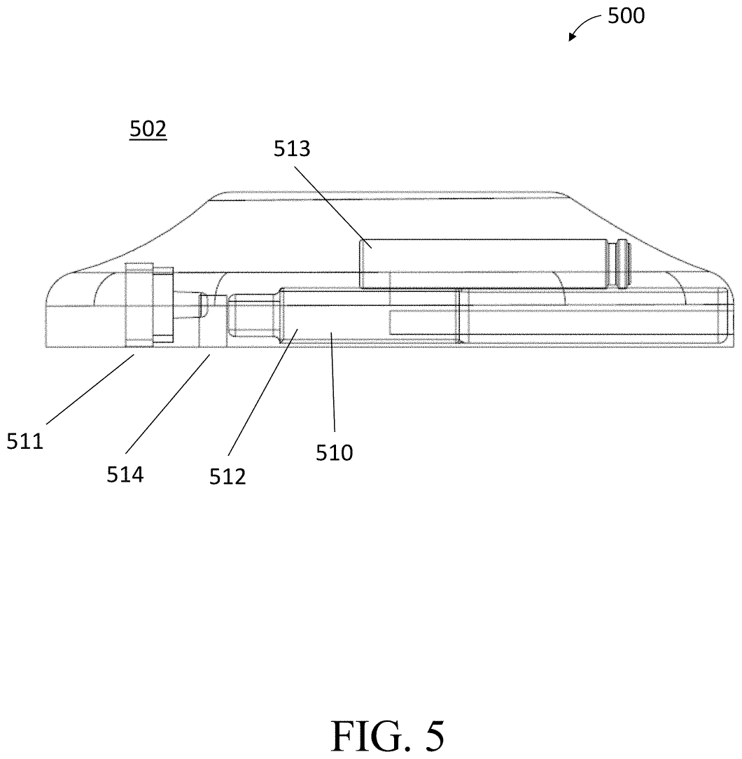

In another exemplary embodiment of the present disclosure and with reference to FIGS. 3A-3C, the NPWT device 100 comprises a pump 302. In some embodiments, the pump 302 comprises an inlet port 314 and an outlet port 315. In some embodiments, pump 315 draws air through the inlet port 314 and expels air through the outlet port 315. Although a portion of pump 315 is shown as protruding from pump 302, the pump 315 may be fully enclosed within the pump 302 or external to a wound dressing without departing from the embodiments contemplated herein.

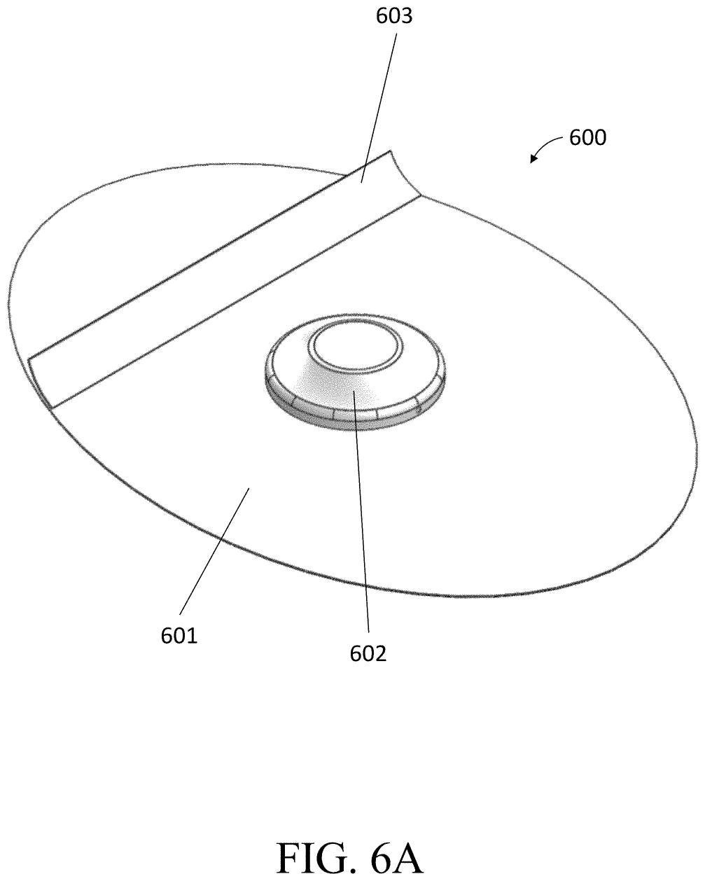

In another exemplary embodiment of the present disclosure and with reference to FIG. 4, the NPWT device 100 comprises a pump 402. The pump 402 may be constructed from any pliable, rigid, or semi-rigid material suitable for constructing such components. For example, some or all of the pump 402 may comprise polymers such as low-density polyethylene (LDPE), high-density polyethylene (HDPE), polypropylene (PP), polyvinyl chloride (PVC), polystyrene (PS), nylon, nylon 6, nylon 6.6, Teflon (polytetrafluoroethylene), thermoplastic elastomer (TPE), thermoplastic polyurethanes (TPU), or any combination thereof. In other embodiments, some or all of the pump 402 may comprise one or more metals or metal alloys. The pump 402 may comprise a pump 410, a plurality of sensors 411, a PCBA 412, a power source 413, an inlet port 414, and an outlet port 415.

In another exemplary embodiment of the present disclosure, the inlet port 414 and/or the outlet port 415 may also be sized or configured to suit the needs of pump 410. In another exemplary embodiment, the outlet port 415 and/or inlet port 414 may be configured to work with additional NPWT devices or accessories. In such an embodiment, the pump 402 may be configured to work with a wide variety of dressings, e.g., dressings 201.

In another exemplary embodiment of the present disclosure, the plurality of sensors 411 comprises a pressure sensor configured to determine the pressure between the NPWT device 402 and the patient's skin, i.e., at the wound or periwound site. In such an embodiment, the sensor 411 conveys pressure information, for example, using the PCBA 412, to the pump 410. In some embodiments, the PCBA 412 is located within or integrated into pump 410. In other embodiments, the PCBA 412 is operatively connected to the pump 410, the plurality of sensors 411, and the power source 413. In another embodiment, the pump 410 is configured to create and maintain the desired amount of vacuum between the NPWT device 402 and the patient. The desired amount of vacuum may be varied without departing from the embodiments contemplated herein. For example, the NPWT device 402 may be configured to create and maintain a vacuum of approximately 60 to 120 mmHg. The desired vacuum may be held at a fixed value or oscillate between a predetermined set of values.

In another exemplary embodiment of the present disclosure, the NPWT device 402 comprises a user control panel (not shown) configured to allow a user to operate and control the functionality of the NPWT device 402. For example, the control panel may comprise a plurality of buttons and/or switches corresponding to various functions of the NPWT device 402. In other embodiments, the NPWT device 402 also comprises an input-output (“I/O”) interface (not shown) configured to connect the NPWT device 402 to other devices. For example, the I/O interface may be configured to wirelessly connect to the NPWT device 402 to patient monitoring equipment. In another embodiment, the I/O interface may be configured to connect the NPWT device 402 to a mobile device that allows the user or patient to control, set, and/or monitor various aspects of the NPWT device 402 using a user interface installed on the mobile device. For example, the patient's mobile device, e.g., smartphone, computer, tablet, or cell phone, may have an application installed thereon that interfaces with the NPWT device 402 through the I/O interface. In such an embodiment, the patient or health care professional may set the amount of vacuum created and maintained by pump 410 and monitor the amount of time that has transpired since the dressing 101 has been changed. In some embodiments, the NPWT device 402 may be configured such that only a health care professional can interface with and control the NPWT device 402. In such an embodiment, the patient is precluded from adjusting the settings of the NPWT device 402. In other embodiments, the NPWT device 402 can be configured to set or broadcast alarms. For example, the NPWT can have timer alarms to alert a user to change the dressing at a predetermined interval. In another embodiment, the NPWT device 402 can broadcast an alarm that signifies that the vacuum applied has fallen out of a predetermined range or limit. For example, the NPWT device 402 can broadcast an alert signifying that the vacuum is below a predetermined threshold, which may indicate that the dressing is inundated with exudate and thus requires changing.

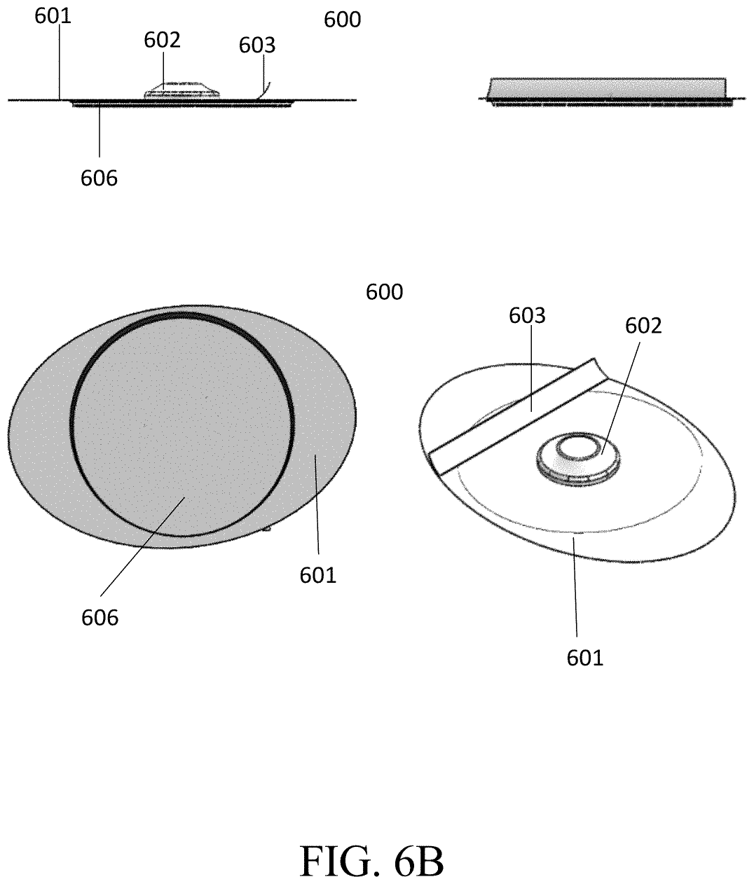

In another exemplary embodiment of the present disclosure and with reference to FIG. 5, the NPWT device 100 comprises a pump 502. The pump 502 may comprise a pump 510, a plurality of sensors 511, a PCBA 512, a power source 513, an inlet port 514, and an outlet port 415. Although the operative components of pump 502 are depicted as being arranged in a certain configuration, the components may be arranged in any configuration, whether fully encapsulated within pump 502 or not, without departing from the embodiments contemplated herein

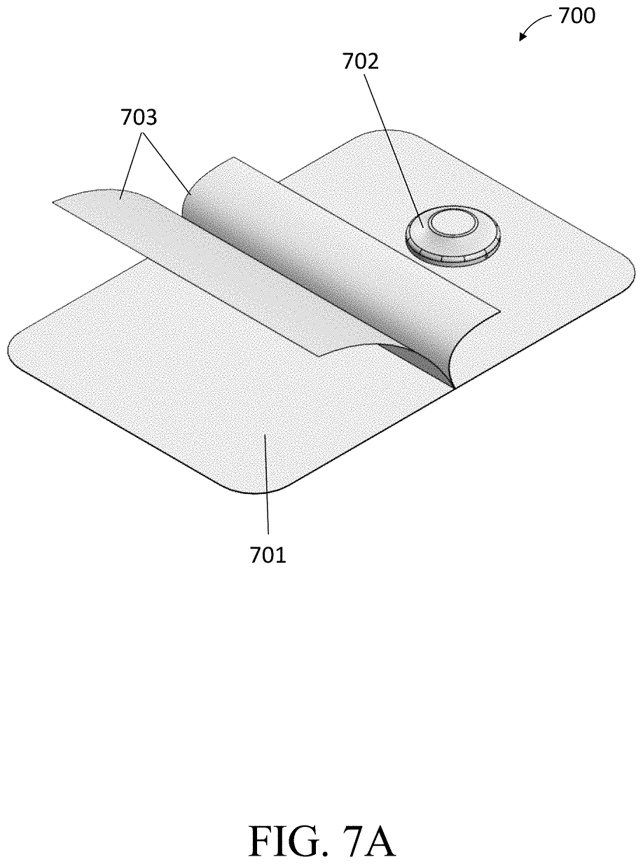

In another exemplary embodiment of the present disclosure and with reference to FIGS. 6A-6B, the NPWT device 600 comprises a dressing 601, a pump 602, a pull tab 603, and a contact layer 606. The dressing 601 can have varying shapes and sizes, for example, an ovular or circular shape.

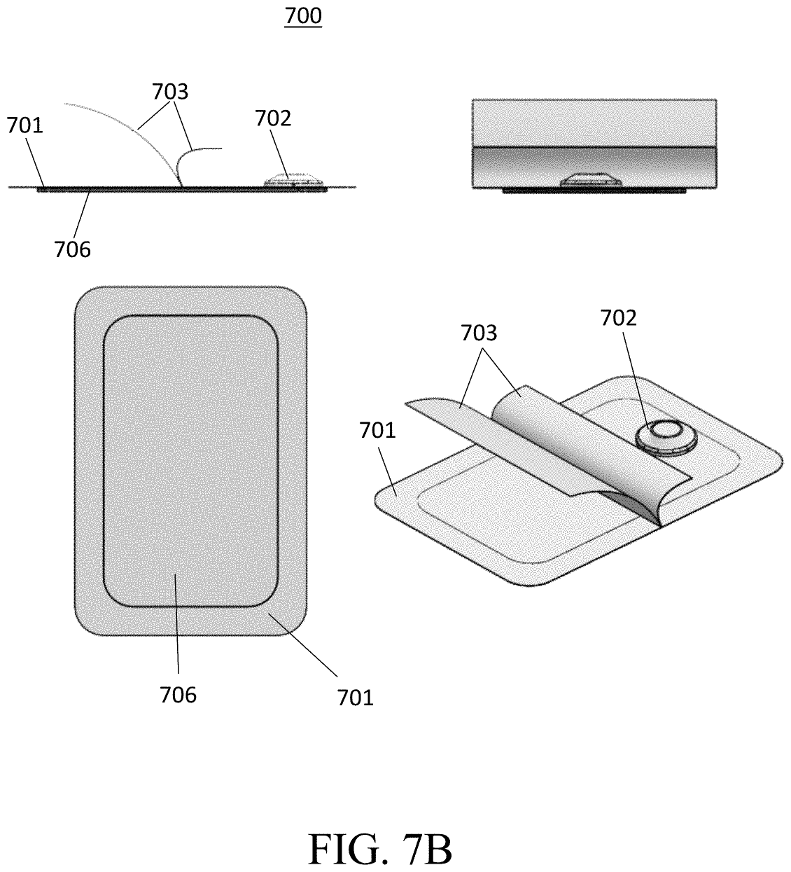

In another exemplary embodiment of the present disclosure and with reference to FIGS. 7A-7B, the NPWT device 700 comprises dressing 701, pump 702, pull tabs 703, and a contact layer 706. The dressing 701 can have varying shapes and sizes, for example, a rectangular shape. Although pump 702 is shown attached to the dressing 701 at a specific location, pump 702 may be attached to the dressing 701 at any suitable location without departing from the contemplated embodiments.

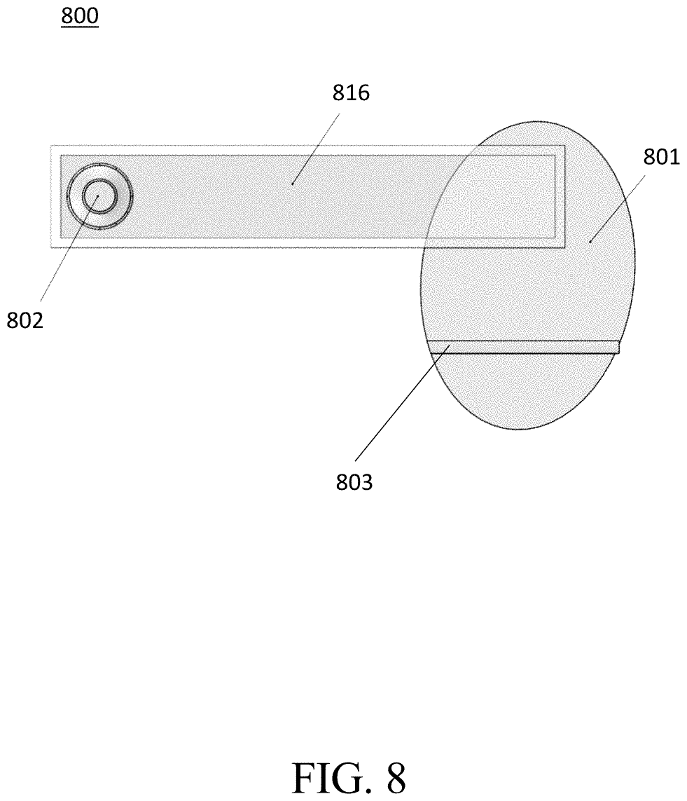

In another exemplary embodiment of the present disclosure and with reference to FIG. 8, the NPWT device 800 comprises a dressing 801, a pump 802, a pull tab 803, and a connector 816. The connector 816 comprises passageways that allow the pump 802 to be located remotely from dressing 801 while still being able to draw air from within the dressing 801. In such an embodiment, connector 816 allows the pump 802 to be located remotely from the dressing 801. Such a configuration allows the dressing 801 to be located in obscure or venerable areas of the patient's body, e.g., hips, shoulders, etc.

The disclosure has been described herein using specific embodiments for the purposes of illustration only. For example, although the various layers of the dressing are shown and described as having a discrete number of layers, any number of layers may be used without departing from the contemplated embodiments. It will be readily apparent to one of ordinary skill in the art, however, that the principles of the disclosure can be embodied in other ways. Therefore, the disclosure should not be regarded as being limited in scope to the specific embodiments disclosed herein but instead as being fully commensurate in scope with the following claims.

1. A negative pressure wound therapy device comprising:
a dressing comprising a drape and a contact layer;
a pump coupled to the drape, wherein the pump comprises a power source, an air inlet in fluidic communication with the contact layer, and an air outlet and the pump, in operation, creates a sub-atmospheric pressure in a region formed by the drape, the contact layer, and the air inlet when the contact layer is affixed to wounded skin; and
a pressure sensor, wherein the pressure sensor triggers an alert that the dressing requires changing when the pressure in the region reaches a predetermined threshold.

2. The device of claim 1, wherein the dressing further comprises an intermediate layer disposed between the drape and the contact layer.

3. The device of claim 2, wherein the intermediate layer comprises one or more hydrophobic materials.

4. The device of claim 2, wherein the intermediate layer comprises one or more hydrophilic materials.

5. (canceled)

6. The device of claim 1 further comprising a pull tab comprising a first end and a second end, wherein the pull tab is attached to the drape at the first end.

7. (canceled)

8. The device of claim 1, wherein the pump is removably attached to the dressing via a coupling.

9. (canceled)

10. (canceled)

11. A method for treating a wound using a negative pressure wound therapy device comprising:
adhering a dressing to a skin area circumscribing the wound, wherein the dressing comprises a drape covering a contact layer;
drawing air from within the dressing using a pump comprising an air inlet in fluidic communication with the contact layer, and a power source, wherein the pump is attached to the dressing and creates a zone of sub-atmospheric pressure in a region formed by between the drape, the contact layer, the air inlet, and the skin area;
sensing an amount of pressure in the region reaching a predetermined threshold; and
generating an alert that the dressing requires changing.

12. The method of claim 11, wherein the dressing further comprises an intermediate layer disposed between the drape and the contact layer; and wherein the pump draws air through the intermediate layer, whereby the intermediate layer captures exudate from the wound.

13. The method of claim 11, wherein the drape comprises a pull tab configured to remove the device.

14. The method of claim 12, wherein the intermediate layer comprises one or more hydrophilic materials.

15. The method of claim 11, wherein the pump further comprises a pressure sensor.

16. The method of claim 11, wherein the pump is removably attached to the dressing.

17. The device of claim 1, wherein the region further comprises a tubeless passageway disposed between and in fluidic communication with the air inlet and contact layer, and the air inlet is coupled to the tubeless passageway and located remotely from the dressing.

18. The method of claim 11, wherein the region further comprises a tubeless passageway disposed between and in fluidic communication with the air inlet and contact layer, and the air inlet is coupled to the tubeless passageway and located remotely from the dressing.
